(19) United States
(12) Patent Application Publication
(10) Pub. No.: US 2007/0167625 A1
(43) Pub. Date: Jul. 19, 2007

(54) Preparation of rosuvastatin

(76) Inventors: Anna Balanov, Rehovot (IL); Natalia Shenkar, Petach Tiqva (IL); Valerie Niddam-Hildesheim, Kadima (IL)

Correspondence Address: KENYON & KENYON LLP, ONE BROADWAY, NEW YORK, NY, 10004, US

(21) Appl. No.: 11/543,357

(22) Filed: Oct. 4, 2006

Related U.S. Application Data
(63) Continuation-in-part of application No. 11/360,725, filed on Feb. 22, 2006.
(60) Provisional application No. 60/655,580, filed on Feb. 22, 2005.
(60) Provisional application No. 60/676,388, filed on Apr. 28, 2005.
(60) Provisional application No. 60/723,491, filed on Oct. 3, 2005.
(60) Provisional application No. 60/723,875, filed on Oct. 4, 2005.
(60) Provisional application No. 60/732,979, filed on Nov. 2, 2005.
(60) Provisional application No. 60/751,079, filed on Dec. 15, 2005.
(60) Provisional application No. 60/760,506, filed on Jan. 19, 2006.
(60) Provisional application No. 60/762,348, filed on Jan. 25, 2006.

(51) Int. Cl. C07D 239/02

(52) U.S. Cl. 544/332

(57) ABSTRACT

Provided are processes for preparing intermediates of rosuvastatin and their use in preparation of rosuvastatin and rosuvastatin salts thereof.

### RELATED APPLICATIONS

This application is a continuation-in-part of U.S. application Ser. No. 11/360,725, filed Feb. 22, 2006, which claims the benefit of U.S. Provisional Application Nos. 60/655,580, filed Feb. 22, 2005; 60/676,388, filed Apr. 28, 2005; 60/723,491, filed Oct. 3, 2005; 60/723,875, filed Oct. 4, 2005; 60/732,979 filed Nov. 2, 2005, 60/751,079, filed Dec. 15, 2005; 60/760,506, filed Jan. 19, 2006; and 60/762,348, filed Jan. 25, 2006, the disclosures of all of which are incorporated by reference in their entirety herein.

### FIELD OF THE INVENTION

The invention is directed to processes for preparing intermediates of rosuvastatin and their use in preparation of rosuvastatin and rosuvastatin salts thereof.

### BACKGROUND

Complications of cardiovascular disease, such as myocardial infarction, stroke, and peripheral vascular disease account for half of all deaths in the United States. A high level of low density lipoprotein (LDL) in the bloodstream has been linked to the formation of coronary lesions which obstruct the flow of blood and promote thrombosis. [See Goodman and Gilman, The Pharmacological Basis of Therapeutics, 9^{th} ed., p. 879 (1996)]. Reducing plasma LDL levels has been shown to reduce the risk of clinical events in patients with cardiovascular disease and in patients who are free of cardiovascular disease but who have hypercholesterolemia. [Scandinavian Simvastatin Survival Study Group, 1994; Lipid Research Clinics Program, 1984a, 1984b.]

Statin drugs are currently the most therapeutically effective drugs available for reducing the level of LDL in the blood stream of a patient at risk for cardiovascular disease. This class of drugs includes, inter alia, compactin, lovastatin, simvastatin, pravastatin and fluvastatin.

The mechanism of action of statin drugs has been elucidated in some detail. The statin drugs disrupt the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion of HMG-CoA to mevalonate, which is the rate determining step in the biosynthesis of cholesterol. Consequently, HMG-CoA reductase inhibition leads to a reduction in the rate of formation of cholesterol in the liver. Decreased production of cholesterol causes an increase in the number of LDL receptors and corresponding reduction in the concentration of LDL particles in the bloodstream. Reduction in the LDL level in the bloodstream reduces the risk of coronary artery disease. [J. A. M. A. 1984, 251, 351-74].

Currently available statins include: lovastatin, simvastatin, pravastatin, fluvastatin, cerivastatin and atorvastatin, which are administered in their lactone form, as sodium salts or as calcium salts.

Rosuvastatin (7-[4-(4-fluorophenyl)-6-isopropyl-2-(N-methyl-N-methylsulfonylamino) pyrimidin-5-yl]-(3R, 5S)-dihydroxy-(E)-6-heptenoic acid) calcium, an HMG-CoA reductase inhibitor can lower LDL-cholesterol and triglycerides levels more effectively than first generation statin drugs. Rosuvastatin calcium has the following chemical formula:

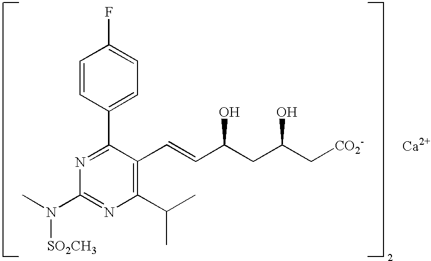

### Rosuvastatin Calcium

A number of relevant processes for preparation of rosuvastatin and salts thereof are disclosed. Rosuvastatin calcium, intermediates and their preparation are disclosed in U.S. Pat. No. 5,260,440, herein '440. WO 03/097614 discloses the synthesis of rosuvastatin from the late intermediate (3R)-3-(tert-butyldimethylsilyloxy)-5-oxo-6-triphenyl-phosphoralydene hexanate, an intermediate disclosed in '440. WO 03/087112 discloses the synthesis of rosuvastatin from a different intermediate, (3R)-3-(t-butyldimethylsilyloxy)-6-dimethoxyphosphinyl-5-oxohexanate. WO/0049014 discloses the synthesis of rosuvastatin using intermediates with other side chains via a Wittig reaction. EP 850,902 describes the removal of triphenylphosphine derivatives in mixtures.

Nevertheless, there remains a need in the art for processes of preparing rosuvastatin that are both cost effective, have fewer purification steps, and/or result in higher purity of the final product, thereby making them more suitable for industrial scale preparation.

### SUMMARY OF THE INVENTION

In one embodiment, the invention provides a process for preparing Compound 20 of the following structure by a Wittig-Homer reaction,

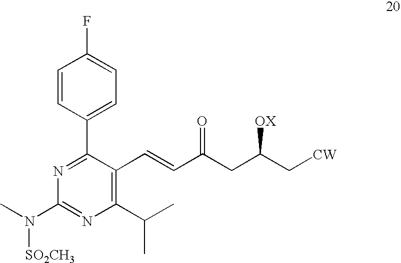

comprising combining Compound 19A of the following structure:

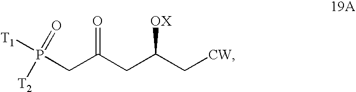

a base and Compound 14 of the following structure:

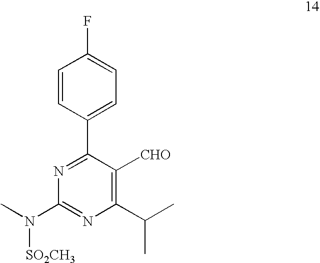

to obtain the Compound 20;

wherein W is a carboxyl protecting group, T1 and T2 are independently aryl or alkoxy, and X is a hydroxyl protecting group.

In another embodiment, the invention provides a process for preparing rosuvastatin or a pharmaceutically acceptable salt thereof, comprising:
a. providing a solution of Compound I of the following structure 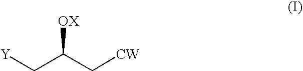
wherein Y is a C₁-C₄ ester, W is a carboxyl protecting group and X is a hydroxyl protecting group, and a polar solvent;
b. combining the solution with a base to obtain a pH of about 10 to about 13 to form a first solution comprising Compound 17 of the following structure 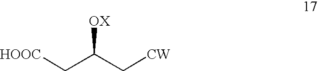
wherein W is a carboxyl protecting group and X is a hydroxyl protecting group;
c. adding a second solution comprising a mono-, di-, tri-(C1 to C4) alkyl substituted benzene chloroformate, saturated or aromatic C5-C12 chloroformate or C1-8 alkyl chloroformate and an organic solvent to obtain a first reaction mixture while maintaining a temperature of about −50° C. to about −10° C.;
d. maintaining the first reaction mixture for a sufficient period of time to obtain Compound 18 of the following structure 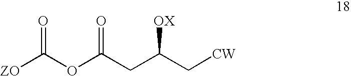
wherein W is a carboxyl protecting group, X is a hydroxyl protecting group and Z is a C₁-₈ alkyl or aryl;
e. providing a dry solvent and Compound 19A of the following structure 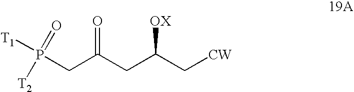
wherein W is a carboxyl protecting group, T₁ and T2 are independently aryl or alkoxy, and X is a hydroxyl protecting group;
f. combining a base with the dry solvent and the Compound 19A to obtain a second reaction mixture;
g. combining Compound 14 with the second reaction mixture at a reduced temperature to obtain a third reaction mixture; 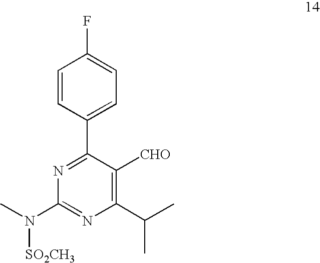
h. maintaining the third reaction mixture for a sufficient time to obtain the Compound 20; 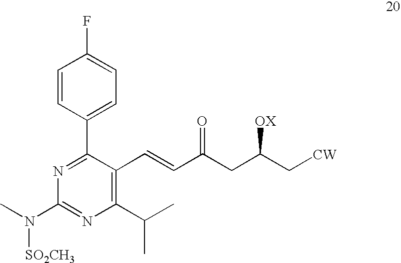
wherein W is a carboxyl protecting group and X is a hydroxyl protecting group;
i. optionally, quenching the reaction;
j. converting Compound 20 into Compound 21 of the following structure 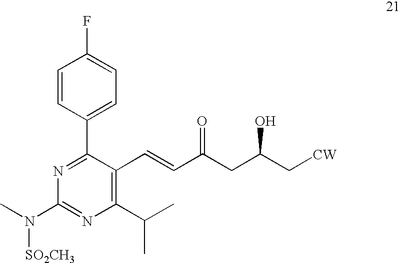
wherein W is a carboxyl protecting group;
k. optionally recovering Compound 21 by providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to Compound 21 and crude Compound 21, washing the non-polar phase with a mixture of lower aliphatic alcohol and water, and recovering Compound 21 from the organic phase;
l. optionally crystallizing Compound 21;
m. converting Compound 21 into Compound 22 of the following structure 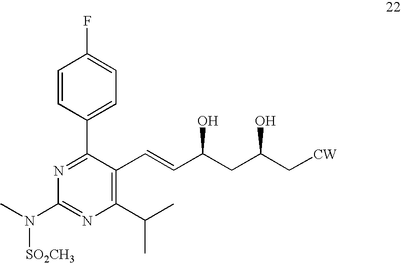
wherein W is a carboxyl protecting group; and
n. converting Compound 22 into rosuvastatin.

### DETAILED DESCRIPTION

As used herein, RT refers to room temperature and includes temperatures of about 25±5° C.

As used herein, “dry solvent” is meant to include any solvent which contains substantially no water, preferably less than 0.5% water.

As used herein a “reduced temperature” is meant to indicate a temperature of less than about 25±5° C.

As used herein, unless otherwise noted, “substantially pure” is meant to indicate a purity of at least about 80%, preferably at least about 85%, and more preferably at least about 95% pure by weight as measured by assay against standard.

The carboxyl protecting group in the structures within the present application may be any suitable carboxyl protecting group, such as esters, amides, benzenes or hydrazides. More preferably, the carboxyl protecting group is an ester, and most preferably is a tert-butyl ester in the structures of the present inventions. Some typical examples of a hydroxyl protecting group include methoxymethyl esters, tetrahydropyranyl ether, trimethylsilyl ether, tertbutyl diphenyl silyl, Stannum derivatives, and acetate ester. Preferably the tri(C₁-C₆ alkyl)silyl is tri(C₁ to C₄ alkyl)silyl, even more preferably trimethylsilyl, or tert-butyldimethylsilyl (TBDMS), with TBDMS being especially preferred. More carboxyl or hydroxyl protecting groups are described in “Protective Groups in Organic Synthesis” by T. W. Greene, John Wiley & Sons, Inc. (1981).

As used herein, “lower aliphatic alcohols” include C₁ to C₄ alcohols.

When used herein, the suffix “TB” describes intermediate compounds described in the summary, wherein R is t-butyl. For example, the term “17TB” refers to intermediate Compound 17 wherein R is t-butyl. The suffix “M” describes intermediate compounds wherein R is methyl. For example, the term “17M” refers to intermediate Compound 17, wherein R is methyl. The suffix “TBPH” describes compounds herein wherein R is t-butyl and PH is phenyl. The suffix “TBRE” describes compounds herein wherein R is tert-butyl and RE is rosuvastatin ester. The suffix “TBDMS” describes compounds herein wherein R is t-butyl and TDMS is tert-butyl dimethyl silyl.

As used herein, “aryl”, “aryl group” or “Ar” refer to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7yl, and the like) provided that the point of attachment is through an aromatic ring atom. Preferably, the aryl is phenyl, naphthyl or 5,6,7,8-tetrahydronaphth-2-yl. The aryl may be substituted or unsubstituted. The substituents may be, for example, an alkyl group, an alkenyl group, a cyclic alkyl group, an aralkyl group, a cyclic alkenyl group, a cyano group, an aryl group, an alkoxy group, an aryloxy group, an alkylthio group, an arylthio group, an alkylsulfonyl group, or an arylsulfonyl group.

The invention provides improved processes for the preparation of rosuvastatin and intermediates thereof in high yield using cost effective reagents. The processes of the invention provide for the quantitative conversion of reagents and decreased formation of by-products, resulting in a process for preparing rosuvastatin requiring fewer purification steps. Examples in specific cases are dispersed throughout.

In one aspect of the present invention, a process is provided for preparing intermediate Compound 17, of the following structure: 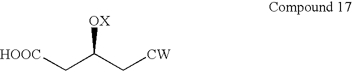
by partial hydrolysis of the diester, Compound I, of the following structure: 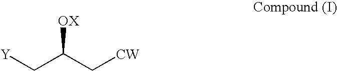
wherein Y is a C₁-C₄ ester, W is a carboxyl protecting group, and X is a hydroxyl protecting group. The process comprises: providing a solution of Compound I and a polar solvent; combining the solution with a base to obtain a pH of about 10 to about 13; and recovering Compound 17. In this process, the synthesis of Compound 17 allows for the production of a monoacid derivative with little contamination of the diacid derivative.

Polar solvents can be selected from the group consisting of: C₁₋₄ alcohols, nitrites, acetone, dioxane, and THF, most preferably, methanol and ethanol. The polar solvent is in amount of about 2 to about 15 volumes, preferably about 5 to about 10, and most preferably 5 volumes relative to Compound I.

The base used is any suitable base, which can be selected from the group consisting of: mono-, di-, tri-(C₁₋₄ alkyl)amino pyridines, mono-, di-, tri-(C₁₋₄ alkyl)amines, alkali metals, alkali earth hydroxides, alkali earth alkooxides, and C₁₋₄ alkyl lithium carbonates. Preferably, the base is at least one of sodium hydroxide, potassium hydroxide, or lithium hydroxide, most preferably sodium hydroxide. Preferably, the base is in a concentration of about 0.9 to about 1.8 volumes, most preferably about 1.2 volumes relative to Compound I.

In a particularly preferred embodiment, the base is added drop-wise to a solution of Compound (I). The base may be added in portions to maintain the pH at this level. The amount of base required to effect the reaction will depend on the scale of the reaction, and may easily be determined by one skilled in the art with little or no experimentation using such techniques as TLC.

Preferably, the reaction mixture is heated at a temperature of about 30° C. to about 70° C. Most preferably, the reaction mixture is heated at about 45° C. to about 55° C. Heating is for a period of time, will depend on scale and mixing procedures, and can be determined by one skilled in the art by measuring the absence of the limiting reagent using such techniques such as HPLC or TLC. For example, when about 288 mmol of Compound I is used, the heating time is about 1 hour to about 10 hours, and preferably about 7 hours.

In another aspect of the present invention, a process for recovery of Compound 17 from the reaction mixture is provided. This process comprises: providing crude Compound 17; partially evaporating the solvent; adding water; washing with a C₅₋₇ alkyl; extracting using an organic solvent selected from the group consisting of: saturated or aromatic C₅-C₁₂ hydrocarbons, mono-, di-, tri-(C₁ to C₄) alkyl substituted benzene; acidifying the mixture using an inorganic acid to a pH of about 7 to about 5; and recovering Compound 17 from the organic phase.

The water used is preferably in an amount of about 2 to about 10 volumes, most preferably 4 volumes relative to the crude Compound 17. Preferably, the C₅₋₇ alkyl is hexane. The washing may be in portions, preferably about 2. The organic solvent is preferably toluene. Any inorganic acid may be used for acidification, preferably HCl. Preferably, acidifying is to a pH of about 6. Recovery from the organic phase may be by drying, such as over MgSO₄.

In another aspect of the present invention, Compound 17 prepared by the process of the present invention is used to prepare any downstream intermediate, rosuvastatin and pharmaceutically acceptable salts thereof by conventional means, for example as depicted in U.S. Pat. No. 5,260,440. For example, the following reaction scheme describes one method of converting Compound 17 into rosuvastatin calcium, wherein Compounds 17 to 22 are represented by number 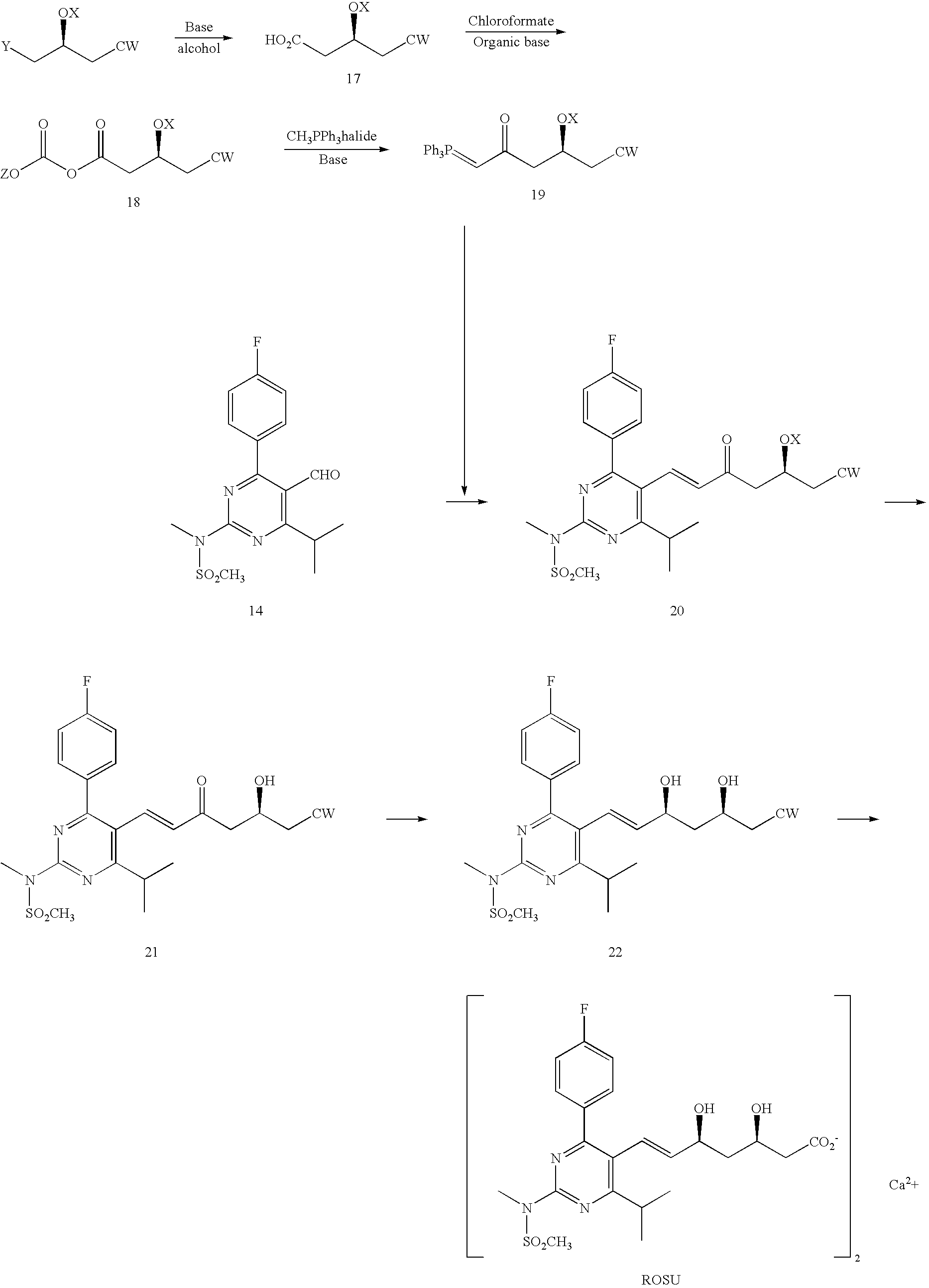
wherein W represents a carboxyl protecting group, Z is a C₁-C₈ alkyl group or aryl, Y is a C₁-C₄ ester, and X is an hydroxyl protecting group.

In one embodiment, in the above scheme, Compound 19 is replaced with Compound 19A, shown below, wherein X and W are as defined above and T1 and T2 are independently aryl or alkoxy. Compound 19A can be prepared from Compound 18 by, e.g., reaction, in a base, of Compound 18 with POQ3, wherein Q is alkoxy or aryl (e.g., (OEt)₂POEt).

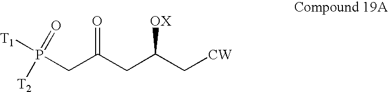

### Preparation of Rosuvastatin through Intermediates

In another aspect of the present invention, a process is provided for preparing intermediate Compound 18, as shown in the following structure: 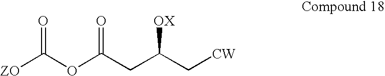
wherein W is a carboxyl protecting group, and X is a hydroxyl protecting group, and Z is a C₁₋₈ alkyl or aryl. The process comprises: adding of a first solution comprising Compound 17, a first organic solvent and a base, to a second solution comprising a mono-, di-, tri-(C₁ to C₄) alkyl substituted benzene chloroformate, saturated or aromatic C₅-C₁₂ chloroformate or C₁₋₈ alkyl chloroformate and a second organic solvent to obtain a reaction mixture while maintaining a temperature of about −50° C. to about −10° C.; and maintaining the reaction mixture for a sufficient period of time to obtain Compound 18.

The base may be any suitable organic base, including, but not limited to, di-(C₁ to C₄ alkyl) pyridine, wherein the alkyl group may be the same or different, mono-, di-, or tri-(C₁ to C₄ alkyl) amines, wherein the alkyl groups can be the same or different, alkaline earth metals, alkaline earth hydroxides, alkaline earth alkoxides, C₁₋₄ alkyl lithium. Preferably, the base is a C₁-₄ trialkylamine, and most preferably is triethylamine.

The first and second organic solvents suitable for use in the process of the invention include, but are not limited to, saturated or aromatic C₅₋₁₂ hydrocarbons, mono-, di-, tri-,(C₁₋₄) alkyl substituted benzenes, and benzenes. For example, THF, toluene, methylene chloride, diethylether, benzene, and chloroform may be used. Toluene and THF are preferred organic solvents. The same organic solvent is preferably used for both the first and second organic solvent.

Preferably the mono-, di-, tri-(C₁ to C₄) alkyl substituted benzene chloroformate, saturated or aromatic C₅-C₁₂ chloroformate or C₁₋₈ alkyl chloroformate is a C₁₋₄ alkyl chloroformate, more preferably ethyl chloroformate or methyl chloroformate, with ethyl chloroformate being particularly preferred. The molar ratio of the chloroformate to Compound 17 in the reaction mixture is about 1 mole to about 3 moles, and is preferably about 1 mol to about 1.5 mol.

The first solution is combined with the second solution at a temperature of about −50° C. to about −10° C., more preferably at a temperature of −50 to about −30° C. and most preferably at a temperature of about −45° C. to about −40° C. Preferably the solutions are combined over a period of about 30 minutes.

The reaction mixture is maintained by gradual heating to about −10° C. to about 30° C., and more preferably to about 0° C. The sufficient period of time required to obtain Compound 18 will depend on, for example, scale and mixing procedures. This can be determined by one skilled in the art by measuring the absence of the limiting reagent using such techniques such as HPLC or TLC, preferably TLC. Optionally, the reaction mixture can then be quenched, preferably with water.

Optionally, Compound 18 may be recovered from the reaction mixture using techniques known to those skilled in the art. Preferably, Compound 18 is recovered by separating the organic layer formed during quenching from the reaction mixture and washing the organic layer with a mild base (pH 7-11), such as NaHCO₃. The reaction mixture may be washed by adding NaCl. The organic layer is then dried, for example with a metal salt, preferably Na₂SO₄ or MgSO₄. The solvent is then evaporated to obtain Compound 18. Alternatively, the reaction mixture is filtered to remove the salts formed during the reaction.

Preparing Compound 18 according to the process of the invention reduces the formation of a symmetric anhydride impurity and allows a quantitative formation of a mixed anhydride product. In addition, the process of this invention can be used easily on an industrial scale as extreme temperatures need not be used, in contradistinction to U.S. Pat. No. 5,260,440 where −70° C. to −85° C. are ideally used.

In another aspect of the present invention, Compound 18 prepared by the process of the present invention is used to prepare any downstream intermediate of rosuvastatin or pharmaceutically acceptable salts thereof.

Compound 18 may be converted into Compound 19 or Compound 19A, of the following structures: 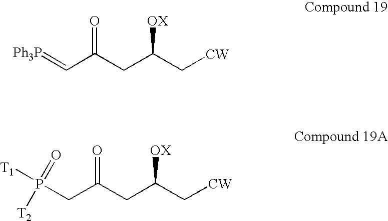
wherein X is any hydroxyl protecting group, T₁ and T2 are independently aryl or alkoxy and W is any carboxyl protecting group, by methods known in the art. For example, a solution of Compound 18 in toluene may be gradually added to a cooled solution comprising: methyl triphenylphosphonium bromide, THF, and a butyllithium while maintaining the temperature at about −60° C. to obtain a reaction mixture; and maintaining the reaction mixture at a maximum temperature of about −20° C. for a sufficient amount of time to obtain Compound 19. [See U.S. Pat. No. 5,260,440].

In another aspect of the present invention, Compound 19 or Compound 19A prepared by the process of the present invention can be used to prepare any downstream intermediate in the synthesis of, e.g., rosuvastatin and pharmaceutically acceptable salts thereof.

In another aspect of the present invention, a process is presented for the preparation of Compound 20. In one aspect, Compound 20 is prepared through the Wittig condensation of Compound 19 and Compound 14, as shown below: 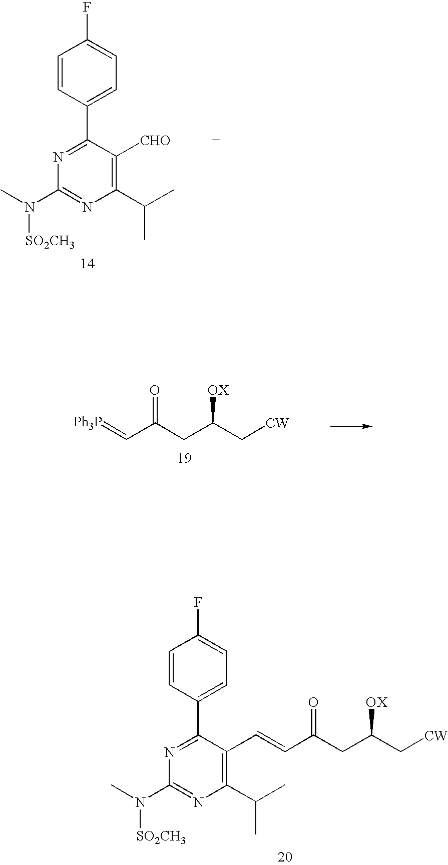
wherein W is a carboxyl protecting group and X is a hydroxyl protecting group. This process comprises: providing Compound 19, Compound 14 and a suitable organic solvent other than acetonitrile, to obtain a reaction mixture in an inert atmosphere such as argon or nitrogen; and heating the reaction mixture at about 70° C. to about reflux for period to obtain Compound 20.

The organic solvent can be any suitable organic solvent including, but not limited to, saturated or aromatic C₅-C₁₂ hydrocarbons, mono-, di-, tri-(C₁ to C₄ alkyl substituted benzenes, and benzenes. Preferably, the organic solvent is toluene.

Compound 19 is in an amount of 1.5 equivalents relative to Compound 14, while the organic solvent other than acetonitrile is about 10 volumes relative to Compound 14. Heating the reaction mixture is preferably to about 70° C. to about 110° C., most preferably about 100° C. The period of time necessary depends on the scale and temperature of the process and may be determined easily by anyone skilled in the art.

Compound 20 may alternatively be prepared by use of a Wittig-Horner reaction (also known as a Horner-Wadsworth-Emmons reaction). (See—Maryanoff et al. “The Wittig olefination reaction”, Chem. Rev. (1989) 89, 863-927; Boutagy et al. “Olefin synthesis with organic phosphonate carbanions”, Chem. Rev. (1974), 74 (1), 87-99; Wadsworth et al.” The utility of phosphonate carbanions in olefin synthesis”, JACS (1961), 83, 1733-1738; Tsuge et al. “Homer-Emmons Olefination”, Bull. Chem. Soc. Jpn. (1987), 60, 4091-4098.) The Wittig-Homer reaction can be applied to Compound 19A and Compound 14, as shown below: 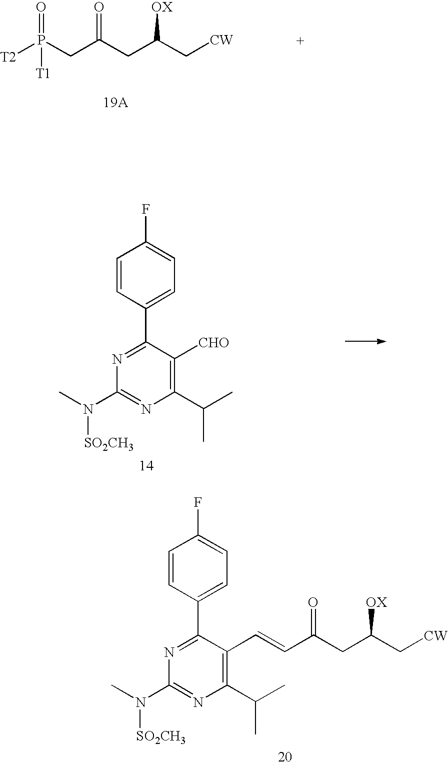
wherein X and W are as defined for Compound 19 and T1 and T2 are independently alkoxy or aryl. For example, Compound 19A may be 19TBPO as shown below.

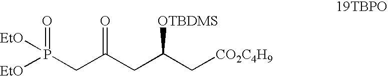

In one aspect of the present invention, a Wittig-Homer reaction is presented which comprises combining Compound 19A, a base, and Compound 14 to obtain Compound 20.

Preferably, the Wittig-Homer reaction for preparing Compound 20 comprises:

(a) providing a dry solvent and the Compound 19A;

(b) combining the base with the dry solvent and the Compound 19A to obtain a first reaction mixture;

(c) combining the Compound 14 with the first reaction mixture at a reduced temperature to obtain a second reaction mixture;

(d) maintaining the second reaction mixture for a sufficient time to obtain the Compound 20.

Compound 19A is, e.g., in an amount of about 1 to 5 molar equivalents relative to Compound 14. Preferably, about 1 to about 2 and more preferably about 1.3 to 1.6 molar equivalents are used.

Examples of dry solvents include, but are not limited to, ethereal solvents such as tetrahydrofuran, aromatic solvents such as toluene, chlorinated solvents and acetonitrile.

Preferably, the dry solvent and Compound 19A are provided in a homogeneous mixture. In one example, the dry solvent and compound 19A are mixed for about 20 minutes to obtain a homogenous mixture. Preferably, the dry solvent and compound 19A are at a temperature below room temperature and above the freezing point of the solvent used, for example, a temperature of 5 to about −5° C.

Preferably combining the dry solvent and Compound 19A is performed at a temperature between about room temperature and about the freezing point of the solvent used, it being noted that the freezing point of any solvent can be easily be obtained by the skilled artisan. Preferably, the dry solvent and Compound 19A are combined until a homogeneous suspension is obtained.

Suitable bases for the Wittig-Homer reaction, include but are not limited to, NaH or other metal hydrides, NaOMe, NaOH, KOtBu, NaOtBu, K₂CO₃, BuLi or other lithiated bases, DBU (1,8-diazabicyclo[5.4.0]undec-7-ene), and DABCO (diazabicyclo[2.2.2]octane). When necessary, suitable bases are in the presence of a phase transfer catalyst. Preferably bases such as lithiated bases, and metal hydrides are used. A sufficient amount of base, for example, is from about 1 to 5 molar equivalents relative to Compound 14, preferably, about 1 to about 2 molar equivalents. Preferably, the base and first reaction mixture are at a reduced temperature of below about 20° C., more preferably below about 10° C., so as to prevent an exothermic reaction. Preferably, the base is added gradually over time.

Preferably, Compound 14 is added gradually over time. Preferably, the temperature is maintained at about less than 20° C., more preferably less than 10° C.

Maintaining the second reaction mixture is preferably for a period of time to allow the reaction to proceed to completion as measured by HPLC. As one skilled in the art will appreciate, the time required to allow the reaction to proceed to completion will vary depending upon, among other factors, the amount of starting materials and the temperature, and can be determined by periodic HPLC measurements. Preferably, more than about 70% of the reaction has gone to completion, more preferably, more than about 85% and most preferably, more than about 95% has gone to completion.

Preferably, once the reaction has gone to completion, quenching of the reaction is performed by addition of water and/or an acid. The acid may be organic or inorganic, strong or weak. For example, acetic acid, hydrochloric acid or ammonium chloride may be used. Preferably, once the reaction is quenched, Compound 20 is recovered.

As assay may be performed which measures contamination of Compound 20 by salts or impurities, primarily from excess of Compound 1 9A and the phosphonate derivative after condensation with the aldehyde (1 equivalent). Regardless of these impurities, Compound 20 formed from this process may be used directly without further purification in the next step to form Compound 21. Recovery may be performed by any suitable means, such as by filtering, washing and drying. In one embodiment, Compound 20 is extracted with a brine solution; the organic phase is washed with a saturated solution of NaHCO₃ and brine solution; and the mixture is evaporated to obtain a viscous oil.

In another embodiment, substantially pure Compound 20 is presented.

In yet another embodiment, a method of recovering Compound 20 is presented comprising:

a. combining the quenched second reaction mixture, which is optionally filtered and washed, with a water immiscible solvent (e.g. hexane, heptane, or toluene) and water to obtain a 2 phase system;

b. washing the first organic [upper] phase with a base (e.g., potassium carbonate (K₂CO₃)) and a solvent (to bring the aqueous base in contact with the organic compound, e.g. an alcohol) to obtain a three phase system; and

c. recovering Compound 20.

In a particularly preferred embodiment, Compound 20 is recovered by a process comprising:

a. combining the quenched second reaction mixture, which is optionally filtered and washed, with a water immiscible solvent and water to obtain an first organic and aqueous phase;

b. washing the first organic phase with a suitable solvent to obtain a second organic and second phase;

c. combining the first organic phase and the second organic phase with a base and an alcohol and optionally adding the extracted product of the first aqueous phase and the second aqueous phase, to obtain a three phase system comprising a upper, middle and lower phase;

d. isolating the upper phase;

e. washing the upper phase with first with an alcohol/water mixture, then a base (e.g. sodium bicarbonate, triethylamine, diisopropylamine, sodium hydroxide), then an alcohol and subsequently water and

f. recovering Compound 20.

The inventors have discovered that the above Wittig-Homer reaction leads to a higher purity downstream products, e.g. Compound 20, as compared to the Wittig reaction.

The by-products obtained from the Wittig-Homer reaction may be separated easily at the end of the reaction after work-up.

Overall, the reaction of Compound 19A with Compound 14 results in a quantitative conversion of starting materials. Preferably, Compound 14 is present in a quantity of less than 5% as measured by HPLC, and most preferably less than 2% as measured by HPLC.

Triphenylphosphine oxide is formed as a by-product of the reactions, and can be removed from the reaction mixture. Preferably, triphenylphosphine oxide is removed by forming a complex with a metal salt by combining a metal salt, preferably anhydrous magnesium chloride with the reaction mixture, as disclosed in EP Patent No. 0850902A1, and isolating Compound 20 by heating to about 100° C., cooling to about 0° C., filtering, washing with water or toluene and evaporating the solvent.

In another aspect of the present invention, Compound 20 prepared by the process of the present invention is used to prepare any downstream intermediate of rosuvastatin and pharmaceutically acceptable salts thereof.

Compound 21 may be prepared by the deprotection of the hydroxyl group of Compound 20, as disclosed in WO 2003/097614 A2 as shown below: 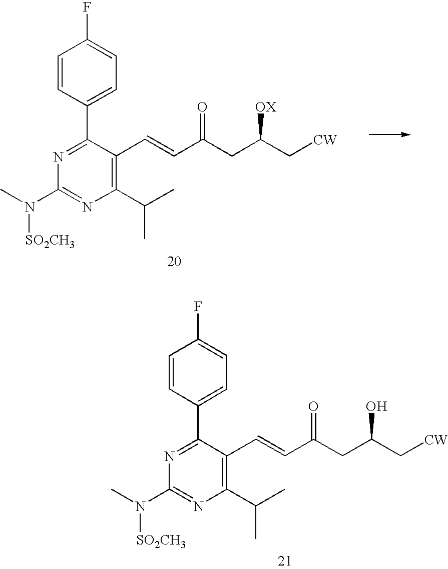
wherein W is a carboxyl protecting group and X is a hydroxyl protecting group. In one example, a solution of Compound 20 in methanol, THF or acetonitrile is combined with a deprotecting agent, such as a fluoride ion source or an inorganic acid aside from HF, to obtain a reaction mixture; and the reaction mixture is maintained for a sufficient time and temperature to obtain Compound 21.

In another aspect of the present invention, a process for recovery of Compound 21 is provided. This process comprises:
a. providing a two-phased system comprised of a mixture of a non-polar aliphatic 15 solvent and a non-polar aromatic solvent and a mixture of a mixture of a lower aliphatic alcohol and water, each in an amount of about 4 to about 6 volumes relative to Compound 21 and crude Compound 21;
b. washing the non-polar phase with a mixture of lower aliphatic alcohol and water; and
c. recovering Compound 21 from the organic phase.

Compound 21, having a purity of greater than about 80%, preferably about 90% (as determined by HPLC) and a yield of greater than about 90%, preferably greater than about 95%, may be obtained using this recovery method.

Preferably, the non-polar aliphatic solvent, non-polar aromatic solvent, lower aliphatic alcohol and water in step a. are each in an equal volume of about 5 volumes relative to Compound 21. Preferably, the non-polar aliphatic solvent is heptane. Preferably, the non-polar aromatic solvent is toluene. Preferably, the lower aliphatic alcohol is ethanol. Preferably, providing the two-phase system of step a. includes mixing the reagents of step a. at room temperature until a clear solvent is obtained and allowing the mixture to separate into phases.

Washing the non-polar phase with the mixture of polar solvent and water is preferably in stages, where 5 times should be sufficient. In a more preferred embodiment, 4 portions of ethanol and water is used. Preferably, the ratio of ethanol to water is in a ratio of about 2:1 by volume. Preferably, the ethanol is in an amount of about 4 to about 6 volumes, preferably 5 volumes relative to Compound 21 while the water is in an amount of about 8 to about 12 volumes relative to Compound 21, preferably about 10 volumes. Preferably, fractions 2 through 5 from 5 fractions are collected, combined and concentrated, preferably under reduced pressure, to obtain an oily residue of Compound 21.

The recovery process of Compound 21 described above allows for the crystallization of Compound 22 after stereoselective reduction of Compound 21. The production of Compound 22 in solid form resulting from the purification of Compound 21 allows rosuvastatin to be further purified, if desired. Crystallization of Compound 21 may further reduce the impurities present; however, such crystallization may not provide a satisfactory yield.

Subsequent reduction of intermediate Compound 21 to form Compound 22, shown in the following: 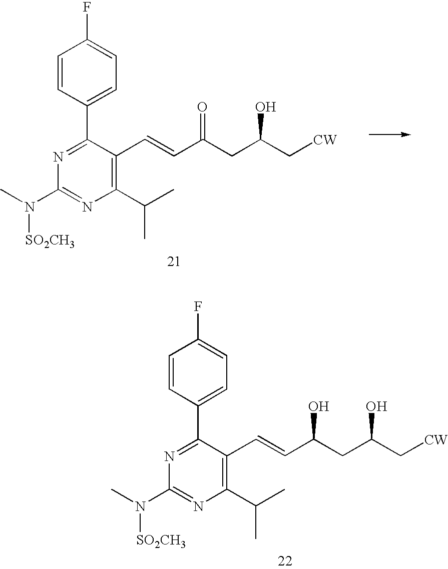
wherein W is a carboxyl protecting group and X is a hydroxyl protecting group. This process is performed under conditions known to those skilled in the art, and is preferably performed using diethylmethoxyborane in THF and sodium borohydride.

Rosuvastatin may be obtained upon saponification of Compound 22.

In another aspect, the present invention provides a process for preparing rosuvastatin, and pharmaceutically acceptable salts thereof, by converting Compound 17 into rosuvastatin.

This process comprises:

a. providing a solution of Compound I and a polar solvent;
b. combining the solution with a base to obtain a pH of about 10 to about 13 to form a first solution comprising Compound 17;
c. adding a second solution comprising a mono-, di-, tri-(C₁ to C₄) alkyl substituted benzene chloroformate, saturated or aromatic C₅-C₁₂ chloroformate or C₁₋₈ alkyl chloroformate and an organic solvent to obtain a first reaction mixture while maintaining a temperature of about −50° C. to about −10° C.;
d. maintaining the first reaction mixture for a sufficient period of time to obtain Compound 18;
e. converting Compound 18 into Compound 19;
f. providing Compound 19, Compound 14 and a suitable organic solvent other than acetonitrile, to obtain a first reaction mixture in an inert atmosphere such as argon or nitrogen;
g. heating the first reaction mixture at about 70° C. to about reflux for period to obtain Compound 20;
h. converting Compound 20 into Compound 21;
i. optionally recovering Compound 21 by providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to Compound 21 and crude Compound 21, washing the non-polar phase with a mixture of lower aliphatic alcohol and water, and recovering Compound 21 from the organic phase;
j. converting Compound 21 into Compound 22; and
k. converting Compound 22 into rosuvastatin.

Optionally, steps e, f, and g are replaced with:

(aa) providing a dry solvent and Compound 19A;

(bb) combining a base with the dry solvent and the Compound 19A to obtain a second reaction mixture;

(cc) combining Compound 14 with the second reaction mixture at a reduced temperature to obtain a third reaction mixture;

(dd) maintaining the third reaction mixture for a sufficient time to obtain Compound 20;

(ee) optionally, quenching the reaction to obtain a fourth reaction mixture comprising Compound 20; and,

(ff) optionally recovering the Compound 20.

Optionally, step (ff) comprises:

(i) combining the quenched second reaction mixture, which is optionally filtered and washed Compound 20, with a water immiscible solvent (e.g. hexane, heptane, or toluene) and water to obtain a 2 phase system;

(ii) washing the first organic [lower] phase with a base (e.g., potassium carbonate (K₂CO₃)) and a solvent (to bring the aqueous base in contact with the organic compound, e.g. an alcohol) to obtain a three phase system; and

(iii) recovering Compound 20.

Optionally step (ff) comprises:

(i) combining the quenched second reaction mixture, which is optionally filtered and washed Compound 20, with a water immiscible solvent and water to obtain an first organic and aqueous phase;

(ii) washing the first organic phase with a suitable solvent to obtain a second organic and second phase;

(iii) combining the first organic phase and the second organic phase with a base and an alcohol and optionally adding the extracted product of the first aqueous phase and the second aqueous phase, to obtain a three phase system comprising a upper, middle and lower phase;

(iv) isolating the upper phase;

(v) washing the upper phase with first with an alcohol/water mixture, then a base (e.g. sodium bicarbonate, triethylamine, diisopropylamine, sodium hydroxide), then an alcohol and subsequently water and (vi) recovering Compound 20.

Optionally, Compound 17 may be recovered from step b. by partially evaporating the solvent from the first solution, adding water, washing with a C₅₋₇ alkyl, extracting using an organic solvent selected from the group consisting of: saturated or aromatic C₅-C₁₂ hydrocarbons, mono-, di-, tri-(C₁ to C₄) alkyl substituted benzene, acidifying the mixture using an inorganic acid to a pH of about 7 to about 5; and recovering Compound 17 from the organic phase. The recovered Compound 17 may then be combined with a first organic solvent and a base to form the first solution comprising Compound 17.

Rosuvastatin obtained by the processes of the invention may be converted to a pharmaceutically acceptable salt of rosuvastatin, preferably the calcium salt. [See e.g. U.S. Pat. No. 5,260,440]. The process of converting rosuvastatin into its pharmaceutically acceptable salt includes contacting rosuvastatin with calcium hydroxide, or with a stronger base such as sodium hydroxide. The base is preferably combined dropwise with a reaction mixture of rosuvastatin at a suitable temperature, such as a temperature of about 25° C.±5° C. The reaction mixture may be washed with a suitable water immiscible organic solvent. Suitable water immiscible organic solvents include, but are not limited to, hydrocarbons; preferably the water immiscible organic solvent is toluene. The water immiscible organic solvent may be removed by phase separation. Remaining water immiscible organic solvent may be removed by distillation of the reaction mixture, preferably at a temperature of about 40° C. to about 45° C. under reduced pressure (below about 50 mmHg).

The reaction mixture may then be combined with an alkali metal, including a source of calcium such as calcium chloride or calcium acetate, to form the salt of rosuvastatin. [See e.g. U.S. Pat. No. 6,777,552]. For example, calcium chloride may be added dropwise to a reaction mixture of rosuvastatin at a suitable temperature, such as a temperature of about 35° C. to about 45° C., and preferably at about 40° C., over a period of about thirty to about ninety minutes. Active carbon may be combined with a reaction mixture of rosuvastatin to remove impurities from the reaction mixture. If active carbon is used during the conversion of rosuvastatin into its pharmaceutically acceptable salt, the active carbon may be used before or after contacting rosuvastatin with an alkali metal.

The conversion of rosuvastatin into its pharmaceutically acceptable salt may also include filtering the reaction mixture. The reaction mixture may be filtered, such as with Synter and Hyflo®, before or after washing with a water immiscible organic solvent.

Other embodiments of the invention encompass pharmaceutical compositions containing rosuvastatin or rosuvastatin salts made by the processes of the invention and methods of making pharmaceutical compositions comprising converting Compound 17 into rosuvastatin or one or more of the above-mentioned intermediates—e.g. Compounds 18, 19, 19A, 20, 21 and 22.

Pharmaceutical compositions of the invention may include excipients. Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, rosuvastatin and any other solid excipients are dissolved or suspended in a liquid carrier such as water, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxyl toluene, butylated hydroxyanisole and ethylenediamine tetraacetic acid may be added at levels safe for ingestion to improve storage stability.

According to the invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs. The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step. The oral dosage form of the invention is preferably in the form of an oral capsule having a dosage of about 5 mg to about 40 mg, more preferably capsules of 5, 10, 20 and 40 mg.

The present invention, in certain of its embodiments, is illustrated by the following non-limiting examples.

All purities mentioned herein refer to a yield per weight quantification, measured by comparing HPLC of the product versus known standard.

### EXAMPLES

### Example 1

Preparation of Compound 17TB

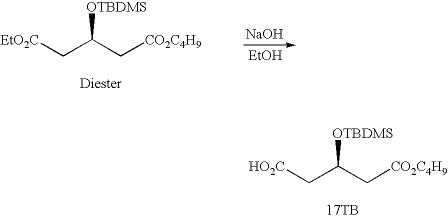

A 1 liter flask, equipped with a condenser, a mechanical stirrer, a pH-meter and a thermometer, was charged with t-butylethyl glutaric acid TBDMS protected (100 g, 288 mmol) and absolute EtOH (500 ml), forming a reaction mixture. The reaction mixture was heated to 50° C., and NaOH 1N (115.2 ml) was added dropwise. The pH measured 12.8.

After 1 hour at this temperature, the pH measured 10.59. Additional NaOH 1N (115.2 ml) was added. The pH measured 12.25. After 1 hour, additional NaOH 1 N (115.2 ml) was added.

The reaction mixture was maintained at 50° C. for 7 hours, until the starting material was not detected by TLC. The reaction mixture was cooled to room temperature, and evaporated to a final volume of 300 ml. H₂O (400 ml) and EtOH (95%, 50 ml) were added to the reaction mixture. The reaction mixture was washed twice with hexane (300 ml each).

Toluene was added (300 ml) to the aqueous phase, and the reaction mixture was neutralized with HCl (32%) to a pH of 6. Two additional extractions with toluene were performed (300 ml each). The toluene layers were combined, dried with MgSO₄ (approx 12 g), and evaporated, yielding 78.3 g (85% yield) of a yellow oil.

### Example 2

Preparation of Compound 18TB

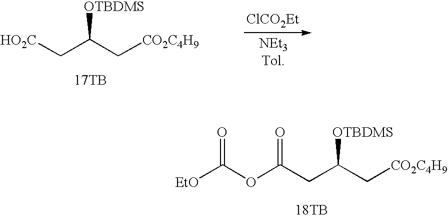

A 2 L flask was charged with a first solution of ethyl chloroformate (16.44 ml) in 900 ml of dry toluene (KF=less than 0.01%) and the solution was cooled to −45° C. A reaction mixture was formed by adding dropwise through a dropping funnel a second solution of Compound 17TB (50 g) and Et₃N (26.06 ml) in 100 ml of toluene dropwise through a dropping funnel to the first solution over a period of about 30 minutes, so that the temperature of the reaction mixture was maintained at −45 to −40° C.

The reaction mixture was slowly heated to 0° C. over a period of 1.5 hours and then quenched with water. The reaction mixture was immediately transferred to a 2L separation funnel, and the organic layer was washed with NaHCO₃ (saturated, 250 ml) and NaCl (saturated, 250 ml), and dried with MgSO₄. The solvent was evaporated and the residue was used for the next stage without any purification.

### Example 3

Preparation of Compound 19TBPH

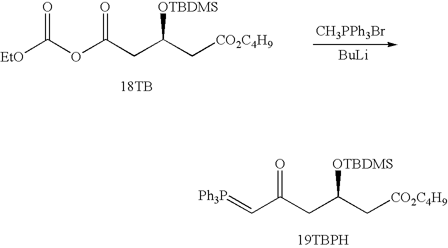

Methyl triphenyl phosphonium bromide (224.3 g) was suspended in THF(600 ml), and BuLi (1.6 M, 392.5 ml) was added over a period of 30 minutes at a temperature of about −55 to −50° C. The reaction mixture was then heated to about 0° C. over a period of 1.5 hours, and then cooled to about −60° C.

A solution of anhydride Compound 18TB (122.6 g, 314 mmol) in toluene (360 ml) was added dropwise to the reaction mixture over a period of about two hours, while the temperature of the reaction mixture was maintained at about −55 to −65 ° C. The reaction mixture was heated to about 0° C. over a period of 1.5 hours, and quenched with water (250 ml). The aqueous phase was separated, and the product was extracted from the aqueous phase using toluene (100 ml). Both organic layers were mixed together and washed with NaHCO₃ (saturated, 2×100 ml) and NaCl (2×100 ml). The organic phase was kept overnight on Na₂SO₄ at about −25° C. and the solvent evaporated before use.

### Example 4

Preparation of Compound 20TB by Wittig Reaction from 19TBPH

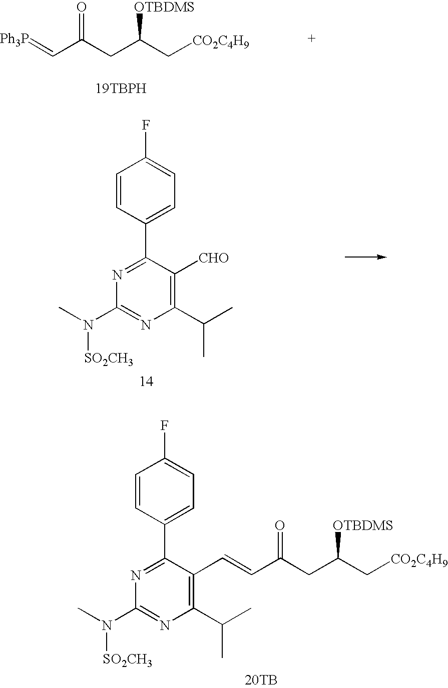

A 100 ml flask, protected from light and provided with N₂ flow was charged with Compound 14 (3.6 g, 10.5 mmol), Compound 19TBPH (9.05 g, 15.7 mmol), and dry toluene (36 ml, 10 vol relative to Compound 14). The reaction mixture was heated to about 100° C. for 19.5 hrs. A sample of the reaction mixture was analyzed by HPLC, and contained 1.7% of Compound 14.

Anhydrous MgCl₂ (2 g, 2 equivalents relative to Compound 19TBPH) was added to the reaction mixture and the reaction mixture was stirred at 100° C. for 2 hrs. The reaction mixture was cooled to 0° C. for 2 hours, and filtered without washing the solid. A filtrate was obtained and was washed twice with H₂O (100 ml each) and the solvent was evaporated, yielding 7.56 g of a brown solid.

### Example 5

Preparation of Compound 20M by Wittig Reaction from 19M

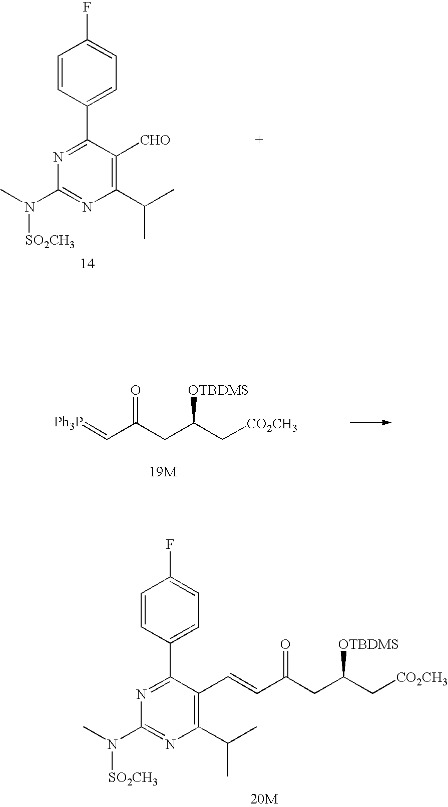

A 250 ml flask, protected from light and provided with N₂ flow was charged with Compound-14 (4.38 g, 12.5 mmol), Compound 19M (10 g, 18.7 mmol), and extra dry toluene (100 ml). The reaction mixture was heated to about 100° C. for 15 hrs. After the completion of the reaction, anhydrous MgCl₂ (4.8 g, 2.7 eq.) was added to the reaction mixture and the reaction mixture was heated for 2 hours at about 100° C. The reaction mixture was cooled to 0° C. over a period of about 2 hours, filtered, and washed with 45 ml of toluene, yielding 12.73 g of a viscous oil.

### Example 6

Preparation of Compound 21TB in HCl/Methanol

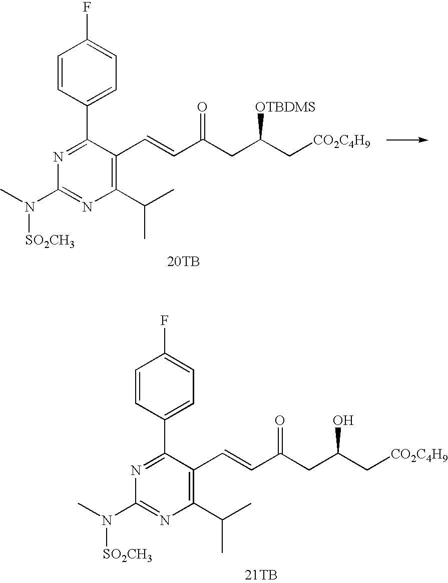

A mixture of HCl (32% in water, 1 mL), water (0.5 mL) and methanol (8 mL) was added dropwise to a solution of Compound 2OTB (2 g) in methanol (10 mL). The reaction mixture was stirred at 30° C. for about 1.5 hours, until TLC (Hexane/EtAc, 4:1) indicated full consumption of the starting material.

Ethyl acetate (150 mL) was added to the reaction mixture and the reaction mixture was washed with a saturated NaHCO₃ solution (50 mL×2), forming an organic layer. The organic layer was dried over MgSO₄ and the solvent was removed under reduced pressure, yielding Compound 21TB (1.72 g).

### Example 7

Preparation of Compound 21TB in HCl/THF

A mixture of HCl (32% in water, 0.57 g), water (2 mL), and THF (17.5 mL) was prepared. 5.4 mL of this mixture were added dropwise to a solution of Compound 20TB (2.7g) in THF (8.1 mL). The reaction mixture was stirred at ambient temperature overnight, until monitoring of the reaction by TLC indicated completion of the reaction.

Ethyl acetate (20 mL) was added to the reaction mixture and the reaction mixture was washed with water (20 mL). An aqueous layer formed, and was extracted with ethyl acetate (20 mL). The organic layers were combined and washed with an aqueous solution of Et₃N (2×5 ml) at a pH of about 10.5. The organic layer was dried over MgSO₄ and the solvent was removed under reduced pressure, yielding an oil of Compound 21TB (2.03 g).

### Example 8

Preparation of Compound 21TB with Tetrabutylammonium Fluoride/THF

Compound 2OTB (5 g) was dissolved in THF (40 mL). Tetrabutylammonium fluoride in THF (8.46 ml, 1 M solution) was added dropwise to the solution, forming a reaction mixture. The reaction mixture was stirred for about 1 hour at room temperature. The solvent was removed under reduced pressure. Toluene (300 ml) was added to the solution. The solution was washed three times with a NaHCO₃ saturated solution (50 mL) and concentrated under reduced pressure, yielding Compound 21TB.

### Example 9

Preparation of Compound 21TB by TBDMS Deprotection with CsF, K₂CO₃ and NH₂OH.HCl

Compound 20TB (0.3 g) was dissolved in acetonitrile (10 ml) at room temperature. CsF (70 mg), K₂CO₃ (300 mg), and NH₂OH.HCl (160 mg) were added to the solution, forming a reaction mixture. The reaction mixture was heated at about 75° C. Partial deprotection of the compound was observed after heating for about 4.5 hours.

### Example 10

Preparation of Compound 21TB by TBDMS Deprotection with CsF

Compound 20TB (300 mg) was dissolved in acetonitrile (10 ml). CsF (70 mg) was added to the solution, forming a slurry. The slurry was heated at about 75° C. for about 17 hours, at which point a complete deprotection of the material was observed.

### Example 11

Preparation of Compound 21TB by TBDMS Deprotection with Tetrabutylammonium Fluoride of 20TB

Compound 20TB (5 g) was dissolved in THF (40 mL) and tetrabutylammonium fluoride was added dropwise as 1 M solution in THF (8.46 mL). The mixture was stirred for 1 hour at room temperature and the solvent was removed under reduced pressure. Toluene (300 ml) was added to the residue. The solution was washed with NaHCO₃ saturated solution (50 mL×3) and concentrated under reduced pressure resulting in crude 21TB.

### Example 12

Preparation of Compound 21TB in Methanesulfonic Acid/Methanol

A solution of methanesulphonic acid (15 mL, 0.2 M in methanol/water, 10:1) was added to a solution of Compound 20TB (3 g) in methanol (15 mL). The reaction mixture was stirred at 30° C. for about 3 hours, until monitoring by TLC (Hexane/EtAc, 4:1) indicated full consumption of the starting material.

Toluene (200 mL) was added to the reaction mixture and the reaction mixture was washed with a saturated NaHCO₃ solution (50 mL×2), forming an organic layer. The organic layer was dried over MgSO₄ and the solvent was removed under reduced pressure to yield Compound 21TB (2.97 g).

### Example 13

Preparation of Compound 21TB by TBDMS Deprotection with Methanesulphonic Acid in Methanol

A solution of methanesulphonic acid (1.66 g) in methanol (200 ml) and water (19 ml) was added to a solution of 20TB (20.26 g, 81.2% assay) in methanol (185 ml). The resulting mixture was stirred at about 30° C. After 10.5 hours the HPLC indicated that the level of the starting material was 6% (on area), and the solution was cooled to room temperature.

EtOAc (400 mL) was added and the solution was washed with brine (400 mL). The organic layer was then washed with a saturated solution of NaHCO₃ (2×200 mL) and finally with brine (2×100 ml).

The organic layer was dried over Na₂SO₄ and the solvent was removed under reduced pressure to obtain 21TB (19.9g).

### Example 14

Preparation of Compound 21M by TBDMS Deprotection with Methanesulphonic Acid in Methanol

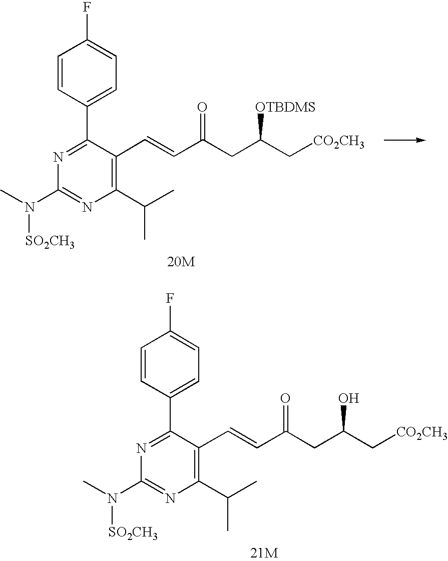

A solution of methanesulphonic acid (50 mL, 0.2 M in methanol/water, 10:1) was added to a solution of Compound 20M (10 g) in methanol (50 mL), forming a reaction mixture. The reaction mixture was stirred at about 30° C. for about four hours. Methanesolfonic acid was added (0.35 ml) to the reaction mixture and the reaction mixture was stirred until completion of the reaction.

A product was extracted with toluene (2×100 mL) and washed with a saturated NaHCO₃ solution (100 mL), forming an organic layer. The organic layer was dried over MgSO₄ and the solvent was removed under reduced pressure, yielding 9.15 g of an oil.

### Example 15

Extraction of Compound 21TB

A 1 L flask equipped with a mechanical stirrer was charged with crude 21 TB (41.6 g, assay=40.8%), toluene (200 mL), ethanol (200 mL), heptane (200 mL), and water (200 mL), forming a suspension. The suspension was stirred at room temperature until a clear solution was obtained. The solution was then poured into a separating funnel to allow phase separation. The EtOH/ H₂O phase was removed. The toluene/heptane phase was then washed 4 times with a mixture of EtOH/ H₂O (400 mL:200 mL), and the fractions were collected. Fractions 2-5 were combined and concentrated under reduced pressure to obtain an oily residue of purified 21TB(24.2 g, assay=56.0%, yield of 80%).

### Example 16

Preparation of Compound 22TB (TBRE)

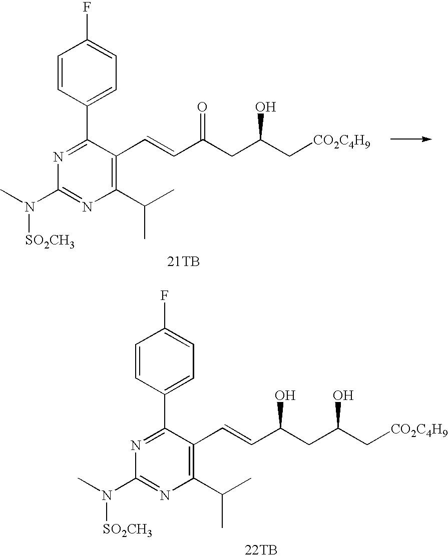

To a solution of 21TB (1 g) in dry THF (26 mL) and dry methanol (7 mL), a solution of diethylmethoxyborane (1 M) in THF (2 mL) was added at about −78° C., forming a reaction mixture. The reaction mixture was stirred for 0.5 hour, NaBH₄ was added, and the stirring was continued for 3 hours. Acetic acid (1.2 mL) was added to the reaction mixture and the reaction mixture was warmed to ambient temperature.

Ethyl acetate (150 mL) was added to the reaction mixture and the pH was adjusted to 8 by addition of concentrated NaHCO₃ water solution. The layers were separated, and water was extracted by adding an additional amount of ethyl acetate (50 mL). The organic layers were combined and dried over MgSO₄. The solvents were then evaporated under reduced pressure, leaving a residue. The residue was treated with methanol and then the methanol was evaporated. Methanol treatment and evaporation was performed two more times, yielding crude Compound 22TB (TBRE) (0.87 g, 86%).

### Example 17

Conversion of Compound 22TB into Rosuvastatin Ca with Extraction in Ethyl Acetate

A 1 L reactor equipped with a mechanical stirrer was charged with EtOH (3 L), water (1800 mL), and TBRE (600 g), forming a reaction mixture. NaOH (47%, 1.2 eq, 114 g) was slowly added to the reaction mixture, at RT. The reaction mixture was stirred at about RT for two hours. The reaction mixture was filtered under reduced pressure with Synter and Hyflo to eliminate the small particles present. The reaction mixture was concentrated under reduced pressure at about 40° C. until half the volume of the reaction mixture remained.

Water (2000 mL) was added to the reaction mixture and the reaction mixture was stirred at about RT for 5 minutes. An aqueous phase and an organic phase formed. The phases were separated, and the aqueous phase was washed with ethyl acetate (3000 mL) and stirred at RT for half an hour. The organic phase was discarded.

The aqueous phase was concentrated under reduced pressure at about 40° C. until half the volume remained. Water (2800 mL) was added to the aqueous phase and the aqueous phase was stirred at about RT for 5 minutes. CaCl₂ (124 g) was added to the aqueous phase in portions over a period of about 10 minutes at a temperature of about RT. The aqueous phase was then stirred at about RT for about 1 hour, filtered, and washed with 1200 mL of water, yielding a powdery compound (491 g, 88%).

### Example 18

Conversion of Compound 22TB into Rosuvastatin Ca with Extraction in Toluene

A 500 mL reactor equipped with a mechanical stirrer was charged with EtOH (150 mL), water (90 mL), and 22TB (30 g), forming a reaction mixture. NaOH (47%, 1.2 eq, 5.7 g) was slowly added to the reaction mixture at a temperature of about RT. The reaction mixture was stirred at RT for about 2 hours. The reaction mixture was filtered under reduced pressure with Synter and Hyflo to eliminate the small particles present. The reaction mixture was washed with toluene (150 mL) and stirred at RT for about half an hour, forming an aqueous phase and an organic phase. The two phases were separated, and the organic phase was discarded.

The aqueous phase was concentrated under reduced pressure at about 40° C. until half the volume remained. Water (104 mL) was added to the aqueous phase and the aqueous phase was stirred at about RT for 5 minutes. CaCl₂ (6.2 g) was added dropwise to the aqueous phase over 1 minute at about RT. The aqueous phase was then stirred at RT for about 1 hour, filtered, and washed with 1200 mL of water, yielding a powdery compound (26 g, 92%).

### Example 19

Conversion of Compound 22TB (TBRE) into Rosuvastatin Ca with Extraction in Toluene

A 1 L reactor equipped with a mechanical stirrer was charged with EtOH (300 mL), water (90 ml), and 22TB (60 g), forming a reaction mixture. NaOH (47% 1.2eq, 11.4 g) was added dropwise to the reaction mixture at RT. The reaction mixture was stirred at about RT for two hours. The reaction mixture was filtered under reduced pressure with Synter and Hyflo to eliminate the small particles present. Water (420 ml) was added to the reaction mixture.

The mixture was then extracted with toluene (3000 mL) and stirred at RT for half an hour. An aqueous phase formed and was isolated. The aqueous phase was concentrated under reduced pressure at 40° C. to half of the volume. Half of the remaining aqueous phase was transferred to a 500 mL reactor and water (110 mL) was added, creating a solution. The solution was stirred at RT for 5 minutes. Ca(OAc)₂ (8.8 g) was added dropwise to the solution over 1 min. at RT. The solution was stirred at RT for 1 hour, filtered, and washed with 60 mL of water, yielding a powdery compound (26 g, 94%).

### Example 20

Synthesis of TB-20 by Wittig-Horner Reaction and Purification Thereof

A 1000 mL 3-necked flask equipped with a mechanical stirrer and nitrogen bubbler was charged with 19TBPO (100 g, 1.5 eq.) and tetrahydrofuran (500 mL). The mixture so-obtained was stirred at 0° C.-2° C. for 20 minutes. Potassium tert-Butoxide (24.7 g, 1.5 eq.) was added in 3 portions while keeping the temperature below 10° C. and the solution was stirred for 15 minutes. ROSU-14 (Compound 14) (51 g, 1.0 eq.) was added and the suspension was further stirred at 0° C.-2° C. for 2 hours. The suspension was then allowed to reach ambient temperature and further stirred for 16-18 hours. Glacial acetic acid (3 mL) was added and the solution was evaporated to dryness to obtain an oily residue.

Heptane (350 mL) and water (250 mL) were added to the oily residue and the organic phase was separated and washed with NaHCO₃ sat. (250 mL). The aqueous phases were combined and extracted with heptane (100 mL). The organic phases were combined and further washed with K₂CO₃ (20%, 350 mL) together with ethanol (350 mL) to obtain 3 phases. The 2 bottom phases were discarded and the remaining organic phase was washed twice with a mixture ethanol (275 mL)/water (275 mL), then again with K₂CO₃ (20%, 275 mL) together with ethanol (275 mL) and finally with water (200 mL). The organic phase was then evaporated to dryness to obtain an oily residue of TB-20. (83.2 g, 82.0% based on 92% assay).

### Example 21

Preparation of Compound 20TB

A 2 L flask under N₂ flow was charged with 19TBPO (100 g, 1.5 eq.) and THF (500 ml, 9.7 vol) and cooled to −5° C. Potassium tert-butoxide (22.97 g, 1.4 eq.) was added through a tube over 15 min. The mixture was stirred for additional 15 min. at this temperature and then Rosu-14 (51.49 g, 1 eq) was added. The bath was removed and the mixture stirred at room temperature for 8 hours.

The reaction was quenched by adding AcOH (6 ml, until pH 5-6). Brine solution (240 ml, 4.6 vol) was added and phases were separated. The organic phase was washed with a saturated solution of NaHCO₃ (300 ml, 5.8 vol) and brine solution (240 ml, 4.6 vol). The organic solvent was evaporated to obtain 136.4 g of a viscous oil. (assay=59.6%, y=81.3%)

### Example 22

Crystallization of Compound 21TB

Oily TB-21 (100 g, assay 65%) was charged in a reactor at ambient temperature with toluene (100 ml). The mixture was stirred until dissolution of all the material (it maybe heated to 50° C. if the oil does not go into the toluene). At ambient temperature hexane (150 ml) was added with a dropping funnel over 30 minutes under slow stirring. A solid started to precipitate after the initial addition of hexane. Additional amount of hexane (250 ml) was added dropwise over 45 minutes at the same temperature. After 3 hrs of stirring, the solid was filtered under reduced pressure, washed with precooled hexane (50 ml, 12-15° C.) and dried under vacuum for 1 hour at about 50° C. to obtain 82 g of a solid material (assay 80%)

### Example 23

Preparation of Compound 22M

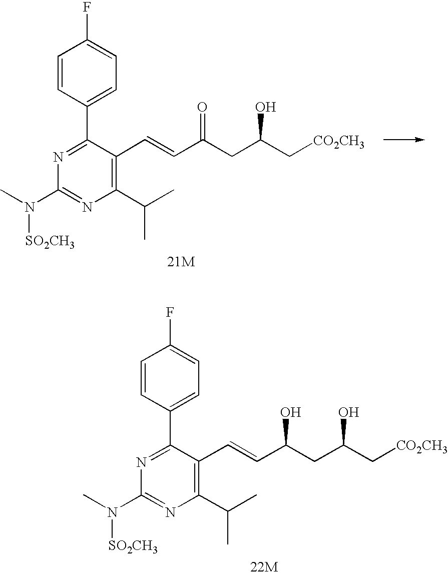

To a solution of Compound 21M (8 g; prepared by a Witig-Horner reaction) in dry THF (210 mL) and dry methanol (56 mL), a solution of diethylmethoxyborane (1 M) in THF (17 mL) was added at −78° C., forming a reaction mixture. The reaction mixture was stirred for 0.5 hour, NaBH₄ (0.76 g) was added, and stirring was continued for 3 hours. Acetic acid (9.6 mL) was added to the reaction mixture and the reaction mixture was warmed to ambient temperature. Water (100 mL), ethyl acetate (100 mL), and a saturated solution of NaHCO₃ were added, forming layers. The layers were separated, and water was extracted with additional ethyl acetate (100 mL). The organic layers were combined and the solvents were evaporated under reduced pressure, leaving a residue. The residue was treated with methanol and methanol was evaporated. Methanol treatment and evaporation was performed two more times, yielding crude compound 22M (4.78 g).

### Example 24

Conversion of Compound 22TB into Rosuvastatin Ca with Extraction in Toluene using Active Carbon

A 1 L reactor equipped with a mechanical stirrer was charged with EtOH (100 mL), water (60 ml), and TBRE (20 g), forming a reaction mixture in suspension. NaOH (47% 1.2 eq, 3.8 g) was added dropwise to the reaction mixture at 25° C.±5° C. The reaction mixture was stirred at about 25° C.±5° C. for two hours. Water (140 mL) was added to the reaction mixture and the reaction mixture was washed with toluene (100 mL). The reaction mixture was stirred at 25° C.±5° C. for half an hour, and then the aqueous phase was isolated.

The aqueous phase was concentrated under reduced pressure at 40° C. to half of its volume. Active carbon was added to the aqueous phase and the aqueous phase was stirred for about half an hour at 25° C.±5° C. The aqueous phase was filtered under reduced pressure with Synter and Hyflo to eliminate the active carbon present. Water (50 ml) was added and the aqueous phase was heated to 40° C. CaCl₂ (4.13 g) was added dropwise to the aqueous phase over a period of about 30-90 minutes at a temperature of about 38° C.-45° C. The aqueous phase was then cooled to 25° C.±5° C., and stirred at 25±5° C. for 1 hour. The aqueous phase was then filtered and washed with water (4×20 ml), yielding a powdery compound (16.7 g dry, 90%).

### Example 25

Conversion of Compound 22TB into Rosuvastatin Ca with Extraction in Toluene using Active Carbon

A 1 L reactor equipped with a mechanical stirrer was charged with EtOH (100 mL), water (60 ml), and TBRE (20 g), forming a reaction mixture in suspension. NaOH (47%, 1.2 eq, 3.8 g) was added dropwise to the reaction mixture at 25±5° C. The reaction mixture was stirred at 25±5° C. for about two hours. Water (140 ml) was added to the reaction mixure, and the reaction mixture was washed with toluene (100 mL). The reaction mixture was stirred at 25° C.±5° C. for half an hour, and then the aqueous phase was isolated.

Active carbon was added to the aqueous phase and the aqueous phase was stirred at 25±5° C. for 30 minutes. The aqueous phase was filtered under reduced pressure with Sinter and Hyflo to eliminate the active carbon present. The aqueous phase was then concentrated under reduced pressure at 40° C. to half of its volume.

Water (50 mL) was added to the aqueous phase, forming a solution. The solution was heated to about 40° C. CaCl₂ (4.13 g) in water (20 ml) was added dropwise to the solution over 30-90 minutes at 38-45° C. The solution was then cooled to 25±5° C., stirred at 25±5° C. for 1 hour, filtered, and washed with water (4×20 ml), yielding a powdery compound (16.7 g dry, 90%).

### Example 26

Conversion of Compound 22TB into Rosuvastatin Ca with Extraction in Toluene using Active Carbon

A 1 L reactor equipped with a mechanical stirrer was charged with EtOH (150 mL), water (90 ml), and TBRE (30 g), forming a reaction mixture. NaOH (47% 1.2 eq, 5.7 g) was added dropwise to the reaction mixture at 25±5° C. The reaction mixture was stirred at 25±5° C. for two hours.

Active carbon was added to the reaction mixture and the reaction mixture was stirred at 25±5° C. for 30 minutes. The reaction mixture was filtered under reduced pressure with Synter and Hyflo to eliminate the active carbon present.

Water (210 ml) was added to the reaction mixture, and the reaction mixture was. washed with toluene (150 mL). The reaction mixture was stirred at 25±5° C. for half an hour, and then the aqueous phase was isolated.

The aqueous phase was concentrated under reduced pressure at 40° C. to half its 5 volume. Water (75 mL) was added to the aqueous phase, forming a solution, and the solution was heated to 40° C.

CaCl₂ (6.2 g) was added dropwise to the solution over 30-90 minutes at 38-45° C. The solution was then cooled to 25±5° C., stirred at 25±5° C. for 1 hour, filtered, and washed with water (4×30 ml), yielding a powdery compound (25 g dry, 90%).

### Example 27

Conversion of Compound 22TB into Rosuvastatin Ca with Extraction in Toluene using Active Carbon

A 1 L reactor equipped with a mechanical stirrer was charged with EtOH (100 mL), water (60 ml), and TBRE (20 g), forming a reaction mixture. NaOH (47% 1.2 eq, 3.8 g) was added dropwise to the reaction mixture at 25±5° C., and the reaction mixture was stirred at 25±5° C. for two hours.

Water (140 ml) was added to the reaction mixture, and the reaction mixture was washed with toluene (100 mL). The reaction mixture was stirred at 25±5° C. for half an hour and the aqueous phase was isolated.

Active carbon was added to the aqueous phase and the aqueous phase was stirred at 25±5° C. for 30 minutes. The aqueous phase was filtered under reduced pressure with Sinter and Hyflo to eliminate the active carbon present.

The aqueous phase was then concentrated under reduced pressure at 40° C. to half its volume. Water (50 mL) was added to the aqueous phase, forming a solution. The solution was heated to 40° C. CaCl₂ (4.13 g) was added dropwise to this solution over 30-90 minutes at 38-45° C. The solution was then cooled to 25±5° C., stirred at 25±5° C. for 1 hour, filtered, and washed with water (4×20 ml), yielding a powdery compound (16.7 g dry, 90%).

What is claimed is:

1. A process for preparing Compound 20 of the following structure by a Wittig-Homer reaction, 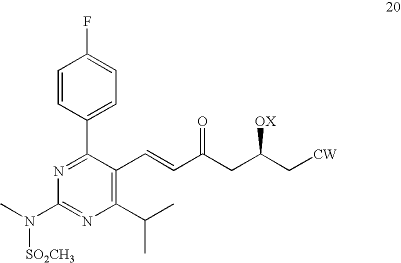
comprising combining Compound 19A of the following structure: 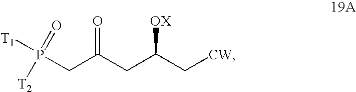
a base and Compound 14 of the following structure: 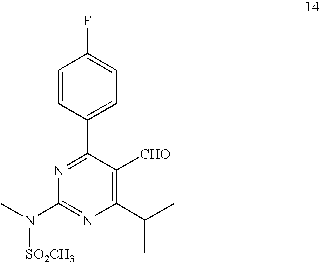
to obtain the Compound 20;
wherein W is a carboxyl protecting group, T1 and T2 are independently aryl or alkoxy, and X is a hydroxyl protecting group.

2. The process of claim 1, wherein the process comprises:
(a) providing a dry solvent and the Compound 19A;
(b) combining the base with the dry solvent and the Compound 19A to obtain a first reaction mixture;
(c) combining the Compound 14 with the first reaction mixture at a reduced temperature to obtain a second reaction mixture;
(d) maintaining the second reaction mixture for a sufficient time to obtain the Compound 20.

3. The process of claim 2, further comprising quenching the reaction after step (d).

4. The process of claim 3, further comprising recovering the Compound 20.

5. The process of claim 4 wherein the recovering comprises:
(i) combining the quenched second reaction mixture with a water immiscible solvent and water to obtain a 2 phase system;
(ii) washing the first organic phase with a base and a solvent to obtain a three phase system; and
(iii) recovering Compound 20.

6. The process of claim 4 wherein the recovering comprises:
(i) combining the quenched second reaction mixture with a water immiscible solvent and water to obtain an first organic and aqueous phase;
(ii) washing the first organic phase with a solvent to obtain a second organic and second phase;
(iii) combining the first organic phase and the second organic phase with a base and an alcohol and optionally adding the extracted product of the first aqueous phase and the second aqueous phase, to obtain a three phase system comprising a upper, middle and lower phase;
(iv) isolating the upper phase;
(v) washing the upper phase with first with an alcohol/water mixture, then a base, then an alcohol and subsequently water; and
(vi) recovering Compound 20.

7. The process of any of claims 5-6 further comprising filtering and washing the Compound 20 prior to the combining the quenched second reaction mixture with a water immiscible solvent and water.

8. The process of claim 3 wherein the quenching comprises adding water and/or an acid.

9. The process of claim 2 wherein the reduced temperature is about room temperature to about the freezing point of the solvent.

10. The process of claim 2 wherein the base is combined in the presence of a phase transfer catalyst.

11. The process of claim 1 wherein the Compound 19A is in an amount of from about 1 to about 5 molar equivalents relative to Compound 14.

12. The process of claim 11 wherein the Compound 19A is in an amount of from about 1 to about 2 molar equivalents relative to Compound 14.

13. The process of claim 1 wherein the base is selected from the group consisting of a metal hydride, NaOMe, KOtBu, NaOtBu, NaOH, K₂CO₃, a lithiated base, 1,8-diazabicyclo[5.4.0]undec-7-ene, diazabicyclo[2.2.2]octane and mixtures thereof.

14. The process of claim 1 wherein the Compound 19A is 19TBPO:

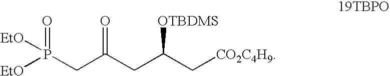

15. A process for preparing Compound 21 of the following structure: 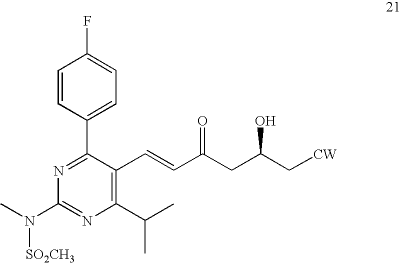
comprising:
i. preparing Compound 20 according to the process of claim 1; and
ii. converting the Compound 20 to the Compound 21;
wherein W is a carboxyl protecting group.

16. A process for preparing rosuvastatin or a pharmaceutically acceptable salt thereof, comprising:
i. preparing Compound 20 according to the process of claim 1; and
ii. converting the Compound 20 to the rosuvastatin or pharmaceutically acceptable salt thereof.

17. A process for preparing rosuvastatin or a pharmaceutically acceptable salt thereof, comprising:
a. providing a solution of Compound I of the following structure 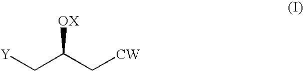
wherein Y is a C₁-C₄ ester, W is a carboxyl protecting group and X is a hydroxyl protecting group, and a polar solvent;
b. combining the solution with a base to obtain a pH of about 10 to about 13 to form a first solution comprising Compound 17 of the following structure 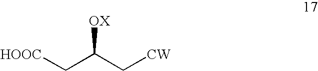
wherein W is a carboxyl protecting group and X is a hydroxyl protecting group;
c. adding a second solution comprising a mono-, di-, tri-(C1 to C4) alkyl substituted benzene chloroformate, saturated or aromatic C5-C12 chloroformate or C1-8 alkyl chloroformate and an organic solvent to obtain a first reaction mixture while maintaining a temperature of about −50° C. to about −10° C.;
d. maintaining the first reaction mixture for a sufficient period of time to obtain Compound 18 of the following structure 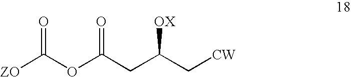
wherein W is a carboxyl protecting group, X is a hydroxyl protecting group and Z is a C₁₋₈ alkyl or aryl;
e. providing a dry solvent and Compound 19A of the following structure 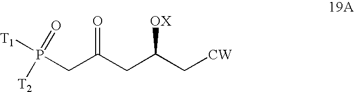
wherein W is a carboxyl protecting group, T1 and T2 are independently aryl or alkoxy, and X is a hydroxyl protecting group;
f. combining a base with the dry solvent and the Compound 19A to obtain a second reaction mixture;
g. combining Compound 14 with the second reaction mixture at a reduced temperature to obtain a third reaction mixture; 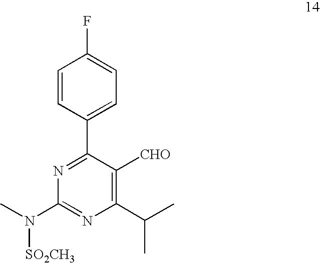
h. maintaining the third reaction mixture for a sufficient time to obtain the Compound 20; 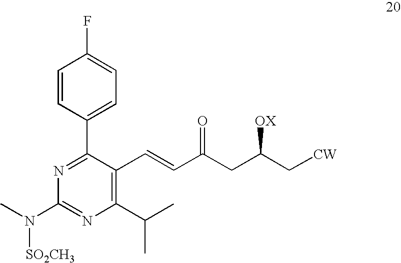
wherein W is a carboxyl protecting group and X is a hydroxyl protecting group;
i. optionally, quenching the reaction;
j. converting Compound 20 into Compound 21 of the following structure 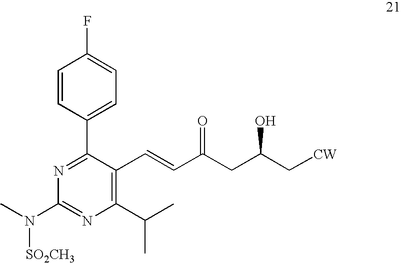
wherein W is a carboxyl protecting group;
k. optionally recovering Compound 21 by providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to Compound 21 and crude Compound 21, washing the non-polar phase with a mixture of lower aliphatic alcohol and water, and recovering Compound 21 from the organic phase;
l. optionally crystallizing Compound 21;
m. converting Compound 21 into Compound 22 of the following structure 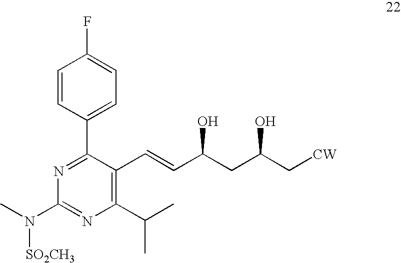
wherein W is a carboxyl protecting group; and
n. converting Compound 22 into rosuvastatin.

18. The process of claim 17 further comprising:
(i) combining the quenched second reaction mixture with a water immiscible solvent and water to obtain a 2 phase system;
(ii) washing the first organic phase with a base and a solvent to obtain a three phase system; and
(iii) recovering Compound 20.

19. The process of claim 17 further comprising:
(i) combining the quenched second reaction mixture with a water immiscible solvent and water to obtain an first organic and aqueous phase;
(ii) washing the first organic phase with a solvent to obtain a second organic and second phase;
(iii) combining the first organic phase and the second organic phase with a base and an alcohol and optionally adding the extracted product of the first aqueous phase and the second aqueous phase, to obtain a three phase system comprising a upper, middle and lower phase;
(iv) isolating the upper phase;
(v) washing the upper phase with first with an alcohol/water mixture, then a base, then an alcohol and subsequently water; and
(vi) recovering Compound 20.

20. The process of claim 17, wherein the rosuvastatin obtained is further converted to a pharmaceutically acceptable salt of rosuvastatin.

21. The process of claim 20, wherein the salt of rosuvastatin is the calcium salt.

22. A pharmaceutical composition comprising rosuvastatin or pharmaceutically acceptable salt thereof prepared according to the process of claim 16 and a pharmaceutically acceptable excipient.

23. A method of lowering cholesterol in a mammal comprising administering the pharmaceutical composition of claim 22 to the mammal.
